(19) United States
(12) Patent Application Publication
(10) Pub. No.: US 2009/0171201 A1
(43) Pub. Date: Jul. 2, 2009

(54) METHOD AND APPARATUS FOR REAL-TIME HEMODYNAMIC MONITORING

(76) Inventor: Eric S. Olson, Maplewood, MN (US)

Correspondence Address: ST. JUDE MEDICAL, ATRIAL FIBRILLATION DIVISION, 14901 DEVEAU PLACE, MINNETONKA, MN, 55345-2126, US

(21) Appl. No.: 11/967,788

(22) Filed: Dec. 31, 2007

(51) Int. Cl. A61B 5/02; A61B 8/08

(52) U.S. Cl. 600/438; 600/486

(57) ABSTRACT

The invention relates to an apparatus for monitoring hemodynamic performance of a cardiac chamber. In one embodiment, the apparatus takes real time measurements of the volume and pressure of the cardiac chamber and prepares a PV loop. The apparatus may include an intracardiac echocardiogram catheter with a pressure sensor positioned to measure intracardiac pressure when the distal end of the catheter is deployed in a cardiac chamber. The apparatus may further include control circuitry that receives heart wall surface image data signals from the ultrasound transducer and intracardiac pressure data signals from the pressure senor and generates pressure-volume loop data signals from the surface image data signals and intracardiac pressure data signals in real time.

### BACKGROUND OF THE INVENTION

a. Field of the Invention

The instant invention relates to medical imaging with catheter based sensors. In particular, the instant invention relates to a catheter system for measurement of cardiac chamber volume or collection of intra-cardiac pressure waveform data to implement a pressure volume loop system.

b. Background Art

Pressure-volume (PV) loops have become popular tools to characterize aspects of the performance of the heart. PV loops are derived from pressure and volume information found in the cardiac cycle. To generate a PV loop for the left ventricle, for example, the left ventricular pressure (LVP) is plotted against left ventricular (LV) volume at multiple time points during a complete cardiac cycle. When this is done, a PV loop is generated. Catheters designed to provide PV loops are known in the art, including those designed and manufactured by Millar Instruments. However, these catheters typically generate volume data by electrical impedance. This data can be inaccurate.

Intracardiac echocardiography (ICE) is a common imaging modality. In recent years, ICE has been used to obtain high-resolution images from within the confines of fluid filled organs and channels. The physics of ICE are the same that are used for all applications of ultrasound. These generally include waves with frequencies greater than 20,000 Hz; sound wave reflection and refraction while crossing borders between materials of different densities; and the use of these waves from and to miniaturized transducers and techniques to create images. These images can be displayed as M and B modes, with Doppler Effect (pulsed wave, continuous wave, or color flow imaging), and as three-dimensional reconstruction.

Two-dimensional ICE is used in today's electrophysiology laboratory because it provides meaningful, real-time anatomic information occurring within the structures of the heart. The catheter-tipped echo transducer typically uses either a series of crystals or a single crystal in which the beam is moved around a circle. The phased array systems consist of either linear phased arrays (sector shaped images with side-firing arrays), or circular phased arrays (radially arranged crystals around the tip of the catheter with a circular image format).

Full Doppler capabilities are now possible with ultrasonic catheters and include pulsed and continuous wave Doppler, color flow Doppler, tissue Doppler, etc. Even newer technologies include pulse inversion, harmonic imaging, strain-rate imaging, and intermittent paging.

Within ablation procedures for atrial fibrillation, for example, ICE imaging allows for the direct visualization of the pulmonary veins, location of the atrial-venal junction and assurance of the ablation catheter tip location within the pulmonary vein antrum. ICE also permits continuous monitoring for radiofrequency (RF) energy delivery during ablation, hemodynamic performance of the myocardium and pericardial space monitoring. For transseptal puncture procedures, right atrial ICE catheter positioning provides clear visualization of the fossa ovalis, tenting by the transseptal catheter and presence of saline bubbles in the left atrium once penetrated by the Brockenbrough needle.

In the clinic, both two dimensional and three dimensional ICE are used for imaging and hemodynamic examinations. While both 2D and 3D ICE generate image data over time, for the purposes of this application 2D ICE refers to 2D image data, including 2D image data derived over time, and 3D ICE refers to 3D image data, including 3D image data derived over time. Likewise, 2D ICE will produce pixel data and 3D ICE will produce voxel data. Data such as blood pressure, pulse rate, etc. can be measured and plotted against time. Combinations of such data can be displayed graphically as multi-dimensional figures.

### BRIEF SUMMARY OF THE INVENTION

However, it is desirable to provide a system for continuous measurement of cardiac chamber volume with simultaneous collection of intra-cardiac pressure waveform data to implement a PV loop system.

Disclosed herein is a method for generating a pressure-volume loop that includes capturing image data of the heart chamber, processing the image data to produce segmented heart chamber size data, tracking segmented heart chamber size data in real time, measuring the pressure of the heart chamber, tracking the measured pressure data in real time, and generating a pressure-volume loop from the generated volume data and the measured intracardiac pressure data in real time.

The method may include placing a pressure sensor in the heart chamber, for example by guiding a catheter based pressure sensor into the heart chamber. The method may also include generating a volume wave form or generating a pressure wave form.

The image data can be gathered from an intracardiac echocardiographic catheter, which may include a pressure sensor such as a piezoelectric material or a hydrostatic pressure transducer.

Processing captured image data to produce segmented heart chamber size data can be done by automatically segmenting the captured image data to provide a direct real time two-dimensional image of cardiac chamber size, or to provide a direct real time three-dimensional image of cardiac chamber size. The processing can be done by automatically implementing a snake segmentation method, or by automatically implementing a level-set/fast-marching segmentation method.

The pressure-volume loop can be output to a display medium. A baseline pressure-volume loop can also be output to the display medium, where it can be compared to the generated pressure-volume loop. In any case, the pressure-volume loop can be evaluated to determine whether one pressure-volume loop represents a more efficient hemodynamic performance of the heart than the other pressure-volume loop.

Also disclosed is a method for generating a pressure-volume loop with the steps of capturing geometric data of the heart chamber, processing the geometric data to produce segmented heart chamber size data, tracking segmented heart chamber size data in real time, measuring pressure data in the heart chamber, tracking the measured pressure data in real time, and generating a pressure-volume loop from the generated volume data and the measured intracardiac pressure data in real time.

The disclosure includes an apparatus for monitoring hemodynamic performance of a cardiac chamber that includes an intracardiac echocardiogram catheter that with a flexible tubular body having a distal end, an ultrasound transducer disposed proximate the distal end, and a pressure sensor disposed in the flexible tubular body such that the pressure sensor is positioned to measure intracardiac pressure when the distal end of the catheter is deployed in the cardiac chamber, and first and second electrical conductors for electrically connecting the ultrasound transducer and the pressure sensor to external control circuitry. This apparatus can further include control circuitry outside the catheter body that is adapted to receive image data signals from the ultrasound transducer and pressure data signals from the pressure sensor.

The control circuitry can generate pressure-volume loop data signals from the surface image data signals and intracardiac pressure data signals in real time, and can provide the pressure-volume loop data signals to an output medium connected to the control circuitry such that the output medium illustrates hemodynamic performance of the cardiac chamber by displaying a pressure-volume relationship graph.

### BRIEF DESCRIPTION OF THE DRAWINGS

FIG. 1 is a partial view of an exemplary embodiment of an intracardiac echocardiogram catheter.

FIG. 1A is a partial view of the distal end of the intracardiac echocardiogram catheter of FIG. 1.

FIG. 2 is a block diagram illustrating an exemplary system for collecting, recording, processing, and displaying intracardiac signals provided by the intracardiac echocardiogram catheter of FIG. 1.

FIG. 3 is a schematic diagram of the apparatus of FIG. 2 connected to a patient.

FIG. 3a is a schematic diagram of the apparatus of FIG. 2 connected to a patient.

FIG. 4 is a representative screen display showing intracardiac echocardiogram information.

FIG. 5 is a representative screen display showing derived wall echocardiogram information of cardiac chamber size at time t=1.

FIG. 6 is a representative screen display showing derived wall echocardiogram information of cardiac chamber size at time t=2.

FIG. 7 is a representative screen display showing derived wall echocardiogram information of cardiac chamber size at time t=3.

FIG. 8 is a representative screen display showing derived wall echocardiogram information of cardiac chamber size at time t=4.

FIG. 9 is a representative screen display showing derived wall echocardiogram information of cardiac chamber size at time t=5.

FIG. 10 is a representative screen display showing derived wall echocardiogram information of cardiac chamber size in three dimensions.

FIG. 11 is an exemplary time series wave form of clinical intracardiac pressure measurements collected by the apparatus of FIGS. 3 and 3a.

FIG. 12 is an exemplary time series wave form of derived cardiac chamber volume information collected by the apparatus of FIGS. 3 and 3a.

FIG. 13 is a cardiac cycle diagram of an exemplary phase of the intracardiac pressure measurements of FIG. 11 and the derived cardiac chamber volume information of FIG. 12.

FIG. 14 is an exemplary pressure-volume loop derived from pressure and volume information of FIG. 13.

FIG. 15 is a block diagram of an exemplary method for generating a pressure-volume loop in real time with the apparatus of FIG. 2.

FIG. 16 is a block diagram of an exemplary method for generating a pressure-volume loop in post processing mode with the apparatus of FIG. 2.

### DETAILED DESCRIPTION OF THE INVENTION

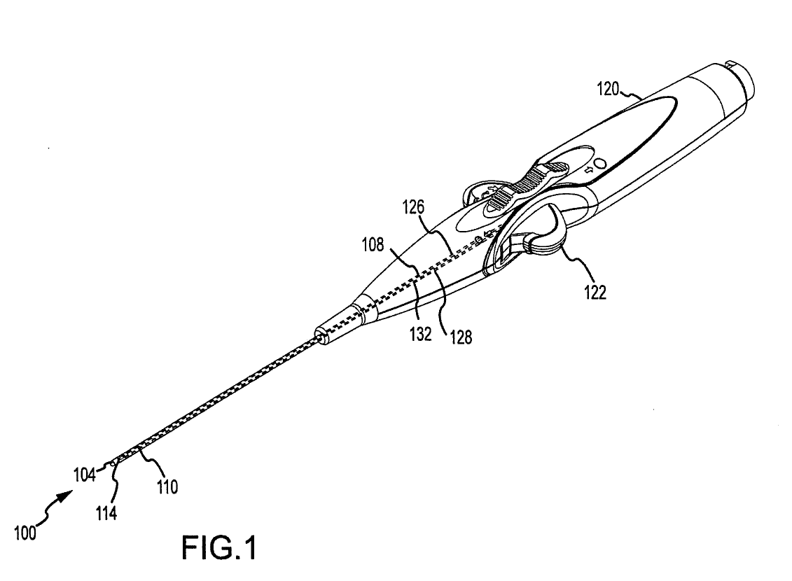

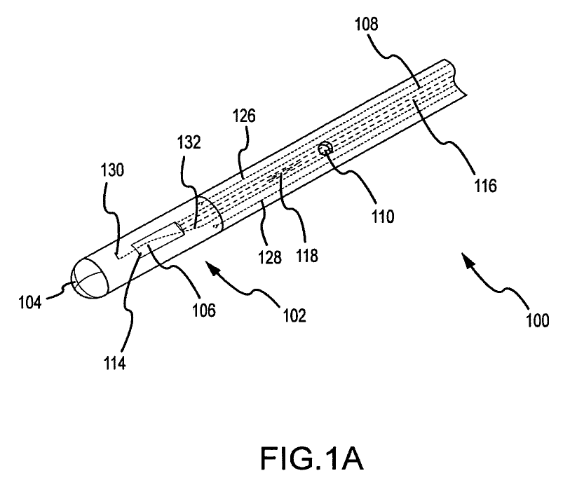

Referring to FIGS. 1 and 1A, catheter 100 includes an elongated flexible tubular catheter body 102 having a distal end 104. Catheter 100 includes near its distal end 104 an ultrasonic transducer 106 which is used to transmit ultrasound and receive resultant echoes to provide a field of view within which flowrates can be measured and features imaged. Transducer 106 can be any known transducer, including but not limited to single element, multiple element, cylindrical element, rotating element, linear array, curved array, circular array, vector phased array, linear phased array, circular phased array or any other mechanical array or dynamic array type that provides a flat two-dimensional tomogram or a volumetric three-dimensional image for intrabody operation within the field of ultrasound. Some examples of such transducers are outlined in N. Bom et al., Early and Recent Intraluminal Ultrasound Devices, ” International Journal of Cardiac Imaging, (1989) Vol. 4, pp. 79-88, incorporated herein by reference herein as though fully set forth herein, while others are known in the art.

An electrical conductor 108 is disposed in catheter body 102 for electrically connecting transducer 106 to control circuitry outside catheter body 102. An access port 110 is configured to allow a pressure sensor 130 within catheter body 102 to measure pressure. Alternatively, the port could allow a temperature sensor or a sensor to determine any other physical parameter within the heart. The flexible tubular body 102 may further define a central lumen 116. The lumen 116 may be a conduit for a suitable biocompatible fluid 118. For example, the lumen may be filled with a saline solution or a radiopaque material

Ultrasonic transducer 106 can be formed using any number of materials that are known in the art. For instance, single crystals, which are capable of operating at a frequency range of, for example, 5 to 50 MHz, are known in the art. Typical materials for forming such crystals include barium titanate or cinnabar. Conductive electrodes such as films of gold or other conductive materials, may be provided on opposing sides of the crystal. If desired, oscillations from the backside of the crystal can be damped through the use of a suitable backing material. Other materials besides piezoelectric crystal oscillators are known for the formation of ultrasonic transducers. For example, organic materials such as polyvinylidene difluoride (PVDF) and vinylidene fluoride-trifluoroethylene copolymers are known, which may also be used to form the ultrasonic transducer 106. The catheter may further include a mechanism for oscillating the transducer 106 in a desired pattern to facilitate scanning the heart. For example, a motor drive may be provided for rotating catheter body 102, although manual rotation may also be employed.

Ultrasonic transducer 106 is located in a distal portion of catheter 100, preferably at or near the distal end 104 of catheter 100. The transducer 106 may be located in a window 114, which provides an aperture through which ultrasonic energy can be directed. Transducer 106 may be located in any fashion known in the art, including behind a lens or cutout. It is also possible to form catheter housing 102 of a material that causes minimal attenuation of the ultrasonic signal transmitted and received by transducer 106. Suitable low-attenuation materials are known in the art and include polyethylene, silicone rubber, polyvinyl chloride, polyurethanes, polyesters, natural rubbers, and the like.

The proximal end of catheter 100 may include a handle 120 with actuator 122 and an electrical plug 124 at the proximal end of the handle 120. The handle and actuator may be of any of the types commonly used in the catheter arts, including the handle disclosed in U.S. Patent Cooperation Treaty application no. PCT/US2007/068269, filed 4 May, 2007, and incorporated herein by reference as though fully set forth herein. Guide wires 126, 128 may extend from handle 120 to any point along catheter 100 for use in controlling the position of a portion of catheter 100 or for controlling the orientation of transducer 106.

The present invention also includes a pressure sensor 130 for sensing the pressure in a subject cavity or lumen. The pressure sensor 130 can be a part of catheter 100, or may be entirely separate, e.g., on a second catheter. In one embodiment, a pressure sensor 130 is located in a distal portion of catheter 100 such as near distal end 104. The pressure sensor 130 may be a piezoelectric sensor. Pressure sensor 130 is connected to an electrical conductor 132, which extends to the proximal portion of the catheter 100. Other types of pressure sensors include transducers and strain gages.

Pressure readings may be taken by other means, including by a pressure sensor external to the catheter or to the body (not shown). In this case the pressure sensor may be in communication with the cavity or lumen through connection to a fluid filled lumen, e.g., lumen 116.

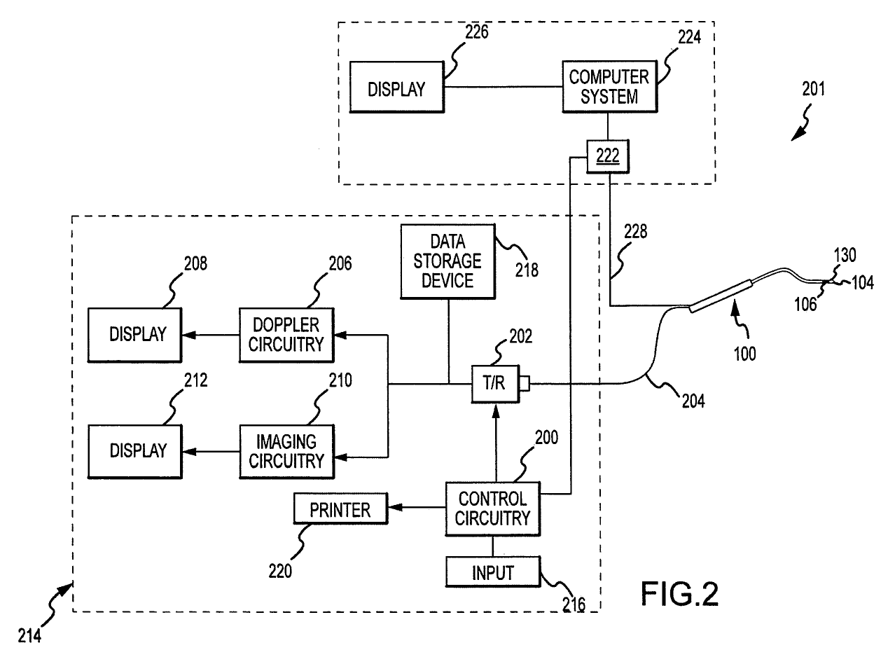

As illustrated in FIG. 2, catheter 100 can be used in a medical system or apparatus 201 that can include parts of two main subsystems. In the first subsystem, a first workstation 214 includes appropriate control circuitry 200 for controlling operation of the ultrasonic transducer. Control circuitry 200 is electrically connected to transceiver circuitry 202 for receiving and transmitting signals via a cable 204 connected to distal handle end 124, and thus is operably connected to electrical conductor 108 and ultrasonic transducer 106. In turn, transceiver circuitry 202 is electrically interconnected to Doppler circuitry 206 and an appropriate display device 208 for displaying physical parameters within the heart. In addition, transceiver circuitry 202 is electrically interconnected to suitable imaging circuitry 210 which is interconnected to a display 212 for displaying images.

Control circuitry 200 causes ultrasonic transducer 106 to vibrate to cause an ultrasonic wave to be emitted from near distal end 104 of catheter 100. The ultrasound wave will propagate through the blood near distal end 104. A portion of the ultrasound wave so emitted will be reflected back from both the moving blood cells and the solid portions of a patient's body to impinge upon transducer 106. An electrical signal is thereby generated and transmitted by cable 108 to transceiver 202. A signal may then be transmitted to Doppler circuitry 206 which will include amplifying and filtering circuitry. Doppler circuitry 206 will analyze the Doppler shift between the transmitted frequency and the received frequency to derive an output proportional to flowrate. This output may then be displayed at display 208 which can be a conventional display terminal. Accordingly, a user is able to obtain a readout of blood flow rates or other hemodynamic information. In addition, a user may be provided with a PV map or other dynamic display.

Similarly, to obtain images, control circuitry 200 triggers ultrasonic transducer 106 through transceiver 202 to produce an ultrasound wave. A portion of the ultrasonic wave is reflected back by features of the patient's body to transducer 106. Cable 108 carries a signal to transceiver 202, which transfers a corresponding signal to imaging circuitry 210 to provide an image of the body features at display 212. Preferably the illustrative control circuitry 210, transceiver 202, Doppler circuitry 206, and imaging circuitry 206 and associated displays 208, 212 are contained in a single workstation 214, but can be separated. Accordingly, the first workstation 214 may include a processor, memory, and associated hardware and software as may be selected and programmed by persons of ordinary skill in the art based on their skill in the art, their goals, and the teachings of the present invention contained herein.

Additionally, the workstation 214 may include a user input device 216 such as a keyboard or a mouse, a data storage device 218 such as a DVD/CD writer for storage and retrieval of data, a hard drive, a networking device or a remote access device. Other components of the workstation 214 may include a printer 220 that allows data to be printed on to paper. Accordingly, the processor, memory and associated hardware and software in the workstation 214 may be selected and programmed to collect, record, process and display signals provided by the intracardiac echocardiogram catheter and other probes and instruments. Additionally, the processor, memory and associated hardware and software in the workstation may be selected and programmed to collect, store or display wave form traces of these signals. Moreover, the processor, memory and associated hardware and software in the workstation 214 may be selected and programmed to create geometric models of cardiac chambers from these signals.

The second subsystem may include a breakout box 222 which preferably provides connections for reference electrodes, probes, the ICE catheter, or therapy catheters. Breakout box 222 includes cable 228 connected to distal handle end 124 (FIG. 1), and thus is operably connected to electrical conductor 132 and pressure sensor 130. Breakout Box 222 connects to computer system 224, which provides information to display 226. The second subsystem can be separate from workstation 214, or it may be combined in one unit.

According to the present invention, image data or geometric data is captured and transmitted, e.g., to the workstation 214 or computer 224 for segmentation to determine and store the heart chamber volume. In addition, the workstation may process the captured image data to produce a geometric model of the cardiac chamber. Although the method and apparatus can utilize an intracardiac echocardiogram catheter to capture volumetric data as described above, other radiological imaging techniques may be used to derive cardiac anatomy and size in real-time. For instance, transthoracic ultra-sound, TEE (trans-esophageal echocardiography), fluoroscopy, rotational angiography, and MRI may be used to continuously measure cardiac chamber volume with simultaneous collection of intracardiac pressure wave form data to implement a pressure-volume loop system.

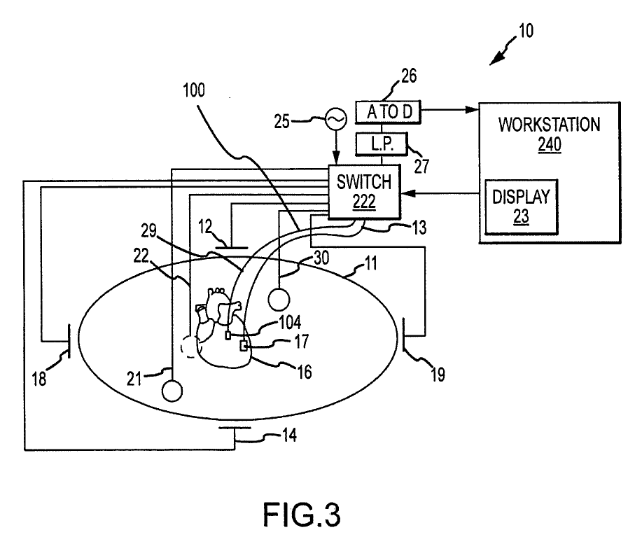

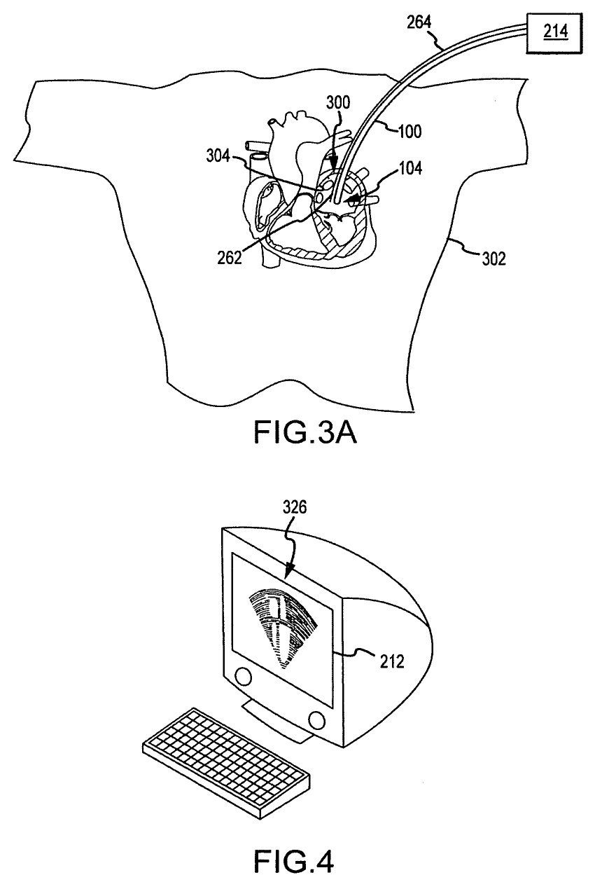

Another method for deriving volumetric information on the heart or a lumen is to utilize the data from an appropriate positioning/location system that allows for internal mapping and modeling. Exemplary positioning/location system include the EnSite® endocardial mapping system produced by St. Jude Medical Inc. and the Carto® system produced by Biosense Webster, Inc. Referring to FIG. 3, in one embodiment, two or more external patient electrode patches 21, 22 are applied on two or more locations on the body. An electrical signal is transmitted between the patches 21, 22, and one or more electrodes of one or more catheters 13 within the heart sense the signal. The system 10 collects electrical data from the catheter(s) 13 and uses this information to track catheter movement and construct models of the heart 16, organ, or lumen in which the catheter is positioned. Additionally a physician may sweep the catheter(s) 13 across the heart chamber during data collection to outline the structures and relay the signals to the computer system, which generates the model. The resulting model may then be utilized to, for example, guide the catheter 13 to one or more locations in the heart where treatment or investigation is needed. Likewise, the resulting model may be utilized to guide distal end 104 or shaft 29 of catheter 100 to locations in the heart.

During mapping, it may be desirable that one of the catheters 13 and/or 100 include a plurality of electrodes for receiving electrical signals. Further, such electrodes may be disposed, for example, in a spiral pattern such that there is a three dimensional displacement of these receiving electrodes. In this arrangement, data gathered by the dispersed three-dimensional array of electrodes may allow for improved mapping of the interior of, for example, a cardiac chamber. Such a system allows for the creation of detailed internal models at the time of study and/or performance of an internal procedure. This is, the system may be operative to generate substantially real-time models.

During catheter ablation, a physician advances the catheter 13 to target tissue utilizing the model. An ablation electrode, for example, catheter electrode 17, is then maneuvered to operationally couple with targeted tissue. Current is then applied to the electrode 17. This current passes through the catheter electrode 17, through patient tissue and back to the external electrode patch 30 on the body surface of the patient. The myocardium around the catheter electrode 17 is heated by Joule effect. When the myocardial temperature exceeds a predetermined threshold (e.g., 50° Celsius) the tissue loses electrical excitability. Stated otherwise, application of electrical energy creates a lesion within the targeted tissue.

In FIG. 3 the patient 11 is depicted as an oval for clarity. In a preferred embodiment, three sets of surface or patch electrodes are shown as 18, 19 along a Y-axis; as 12, 14 along an X-axis; and 21, 22 along a Z-axis. Patch electrode 21 is shown on the surface closest to the observer, and patch electrode 22 is shown in outline form to show its placement on the back of patient 11. An additional patch electrode 30, which may be referred to as a “belly” patch, is also seen in the figure. Each patch electrode 18, 19, 12, 14, 30, 22, 21 is independently connected to a multiplex switch 222. The heart 16 of patient 11 lies between these various sets of patch electrodes 18, 19, 12, 14, 21, 22. Also seen in this figure is a representative catheter 13 having a single distal electrode 17 for clarity. This distal electrode 17 may be a “roving electrode(s)” or a “measurement electrode(s).”

Each patch electrode 18, 19, 12, 14, 30, 22, 21 is coupled to the switch 222, and pairs of electrodes 18, 19, 12, 14, 21, 22 are selected by software running on workstation 240, which with switch 222 couples these electrodes 18, 19, 12, 14, 21, 22 to the signal generator 25. A pair of electrodes, for example electrodes 18 and 19, may be excited by the signal generator 25 and they generate a field in the body of the patient 11 and in the heart 16. During the delivery of the current pulse, the remaining patch electrodes 12, 14, 21, 22 are referenced to the belly patch electrode 30, and the voltages impressed on these remaining electrodes 12, 14, 21, 22 are measured by the analog-to-digital or A-to-D converter 26. Suitable lowpass filtering of the digital data may be subsequently performed in software to remove electronic noise and cardiac motion artifact after suitable low pass filtering in filter 27. In this fashion, the various patch electrodes 18, 19, 12, 14, 21, 22 are divided into driven and non-driven electrode sets. While a pair of electrodes is driven by the signal generator 25, the remaining non-driven electrodes are used as references to synthesize the orthogonal drive axes.

The raw patch voltage data is measured by the A-to-D converter 26 and stored in the second workstation 224 under the direction of software. This electrode excitation process occurs rapidly and sequentially as alternate sets of patch electrodes 18, 19, 12, 14, 21, 22 are selected, and the remaining members of the set are used to measure voltages. This collection of voltage measurements may be referred to herein as the “patch data set”. The software has access to each individual voltage measurement made at each individual patch electrode 18, 19, 12, 14, 21, 22 during each excitation of each pair of electrodes 18, 19, 12, 14, 21, 22. The raw patch data is used to determine the “raw” location in three spaces (X, Y, Z) of the electrodes inside the heart 16, such as the roving electrode 17.

If the roving electrode 17 is swept around in the heart chamber while the heart 16 is beating, a large number of electrode locations are collected. These data points are taken at all stages of the heartbeat and without regard to the cardiac phase. Since the heart 16 changes shape during contraction, only a small number of the points represent the maximum heart volume. By selecting the most exterior points, it is possible to create a “shell” representing the shape of the heart 16, e.g., at its maximum heart volume. The location attribute of the electrodes within the heart 16 are measured while the electric field is impressed on the heart 16 by the surface patch electrodes 18, 19, 12, 14, 21, 22. This shell is then provided to display 23 to provide an image of the heart 16.

The medical navigation/visualization system is also discussed in detail in U.S. Pat. No. 7,263,397, that is entitled “METHOD AND APPARATUS FOR CATHETER NAVIGATION AND LOCATION AND MAPPING IN THE HEART,” that issued on Aug. 28, 2007 and is assigned to the assignee of this patent application, and the entire disclosure of which is incorporated by reference in its entirety herein.

With reference to FIG. 2, in one embodiment, the ultrasound data generated for or by workstation 214 may be displayed on display 226 by second workstation 224, either separately or in combination with the shell created as above.

An exemplary system may also include a generator, such as, e.g., an AC current generator and/or a radio frequency (RF) generator, which provides an electrical signal(s) to the electrode(s) of the catheter 100 as well as to the transducers.

Referring to FIG. 3a, the catheter 100 may be placed in a cardiac chamber 300 of a patient 302 to obtain imaging information 326 (FIG. 4). Catheter 100 maybe placed in the cardiac chamber manually or with robotic assistance. Catheter 100 may also be placed in a luminal area near the heart, or in a pericardial space. Preferably, transducer 106 generates information including image data of the heart chamber 300, and heart chamber wall 304. The captured image data may then be transmitted to the workstation 214 for processing to determine and store the heart chamber volume. For example, the workstation may process the captured image data to produce a geometric model of the cardiac chamber.

Although the method and apparatus can utilize an intracardiac echocardiogram catheter, other radiological imaging techniques may be used to derive cardiac anatomy and size in real-time. For instance, transthoracic ultra-sound, TEE (trans-esophageal echocardiography), fluoroscopy, rotational angiography, and MRI may be used to continuously measure cardiac chamber volume with simultaneous collection of intracardiac pressure wave form data to implement a pressure-volume loop system.

In a preferred embodiment, processing the captured image data includes the automatic segmentation of the images to provide a direct real-time measurement of cardiac chamber size. For instance, in a two-dimensional system this measurement would be area and in a three dimensional system this measurement would be volume. Although, automatic segmentation is preferably implemented by the workstation through the use of deformable models or level-set/fast marching methods other algorithms may be developed or used to achieve automatic segmentation of the image data based on the teachings contained herein. Deformable models comprise such techniques as active Contours (also known as Active Shape Models and Active Template Model) snakes, balloons, etc.

The endocardial surface of the cardiac chamber can be computed over time by active front tracking. This comprises an implicit or explicit representation of the surface. At each time point the surface of the endocardium is evolved to match the updated image data. Once the surface representation of the cardiac chamber has been obtained, the volume of the cardiac chamber can be immediately computed by determining the volume encapsulated by that surface. In a preferred embodiment the segmentation algorithm is the level-set/fast marching method. The chamber is identified by evolving the surface over time. The surface can be represented implicitly as the set of points which satisfy the following relation:

S={ x̅|φ( x̅)=k)

Where,

φ:

[symbol]

[symbol]

[symbol]

is a mapping from the volumetric domain to a scalar value. The set of points which satisfy φ=k (the isovalue) is an isosurface within the volumentric domain. If we consider that the image data is continuously updating and the level function is a function of time, then if we transform the function φ(x,t)=k into a differential equation we have:

$$\frac{\partial φ}{\partial t} = - ∇ φ \frac{d \overline{x}}{d t} ≡ - ∇ φ \cdot v ( t ) ≡ - ∇ φ \cdot F$$

Recognizing dx/dt as the velocity v(t) of the isosurface at a particular location we can define a PDE (partial differential equation) with a “speed function”, F, tuned to performing segmentation. This speed function can be designed to attract the isosurface to characteristics within the image data such as image gradients (i.e. edges or boundaries) while maintaining smooth curvature. One commonly used speed function used for medical image segmentation is:

$$F = α D ( \overline{x} ) + ( 1 + α ) ∇ \cdot \frac{∇ φ}{\vert ∇ φ \vert}$$

Where the speed is a function of the image data D (first term) and the curvature (2^{nd} term). D itself can be a function of the grayscale values of the image as well as the gradient of that image. This speed function is meant to be exemplary as many possible speed functions specialized for particular applications are possible. This PDE can be solved using techniques which are well established in the art.

The invention also encompasses a method of generating data and images utilizing “sectioned” volume data. For example, a 3D image can be divided into angles and the volume of that angle can be computed separately from all the other sections. The sum of all the sections would be the total volume of the chamber. The information provide by a section PV loop may provide unique diagnostic information in such situation where a portion of the cardiac wall is ischemic and not contributing optimally to the performance of the heart. The section may be divided uniformly in angle along the center line of the cardiac chamber (i.e. long axis) or divided in any other manner than may be suitable.

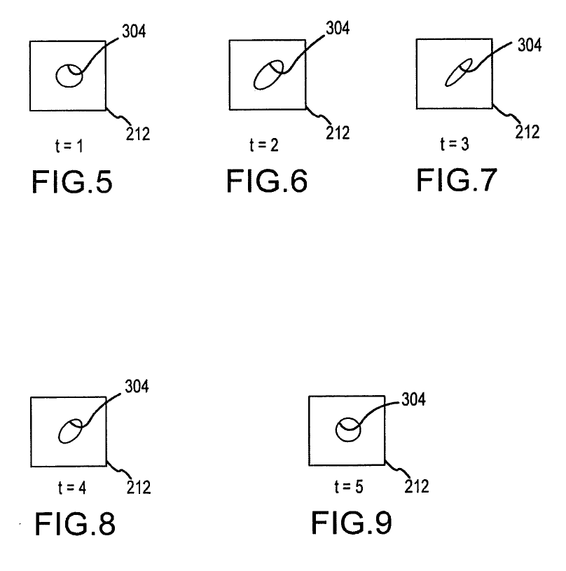

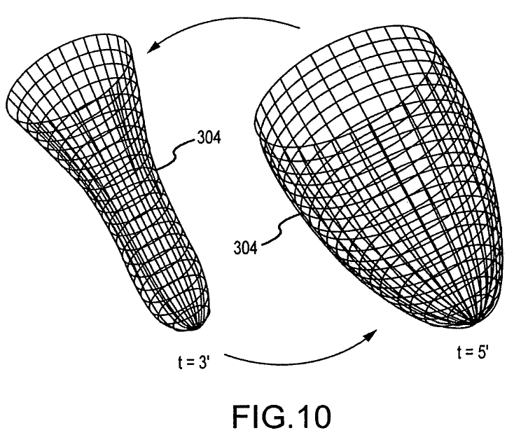

FIGS. 5-9 show exemplary two-dimensional segmented image data of the cardiac chamber boundary 304 over a time series ranging from time t=1 to t=5. By contrast, FIG. 10 shows exemplary three-dimensional segmented image data of the cardiac chamber boundary 304 at time t=3′ and t=5′. From segmented image data the workstation 214 may generate and display a time series volume wave form 260 of cardiac chamber volume as shown in FIG. 12.

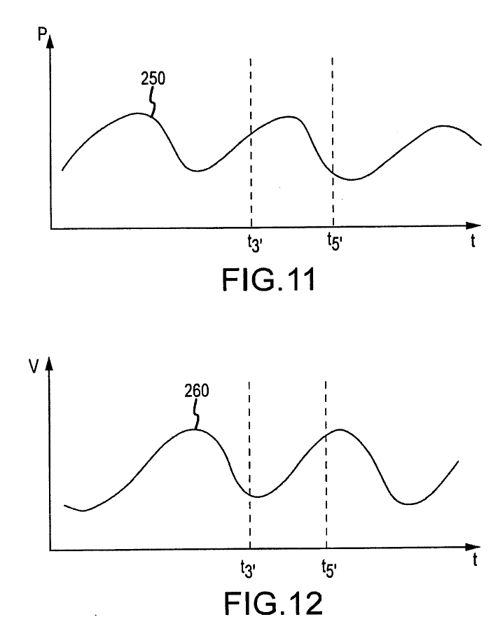

Referring to FIG. 3a, the medical system or apparatus 201 may further include a sensor 262 for measuring intracardiac pressure in the heart chamber 300 where the ICE catheter 100 is disposed. The sensor may be incorporated into a catheter based system 264. Additionally, the pressure sensor may be disposed in the ICE catheter itself as shown in FIG. 1. The pressure sensor may be operated concurrently with the ICE catheter to collect measurements of intracardiac pressure in the heart chamber. The workstation 214 may receive measured intracardiac pressure data signals in real time and store, track, and display intracardiac pressure in a time series pressure wave form 250 as shown in FIG. 11.

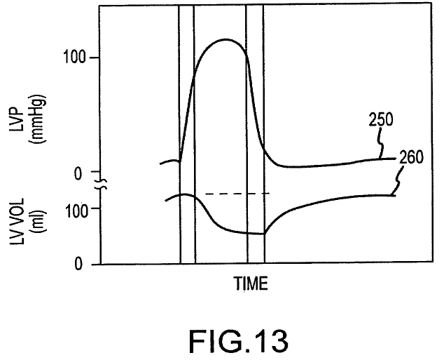

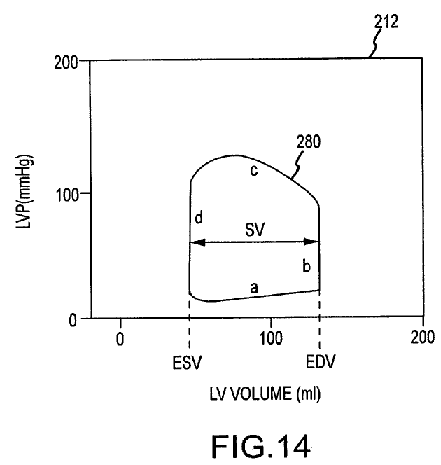

Referring to FIG. 13, an illustrative diagram of pressure and volume data from an exemplary cardiac cycle is depicted. The exemplary pressure and volume data represent a portion of the time series volume wave form 260 and the time series pressure wave form 250 over one cardiac cycle. By contrast, FIG. 14 shows an exemplary pressure-volume (PV) loop 280 derived from pressure and volume information found in FIG. 13. The PV loop 280 has pressure plotted on the ordinate axis and volume plotted on the abscissa.

Referring to FIGS. 3, 3a and 11-14, the medical system 201 may further generate, store, display or otherwise output pressure-volume loop data from the time series volume wave form 260 and the time series pressure wave form 250. For example, P-V loop data for a series of cardiac cycles may be printed or displayed on. display 212. For instance, the printed or displayed information may be a numerical readout, graph, or image data that allows the user to assess the hemodynamic performance of the heart. The hemodynamic performance of the heart may be compared to normal healthy performance or a base-line performance to evaluate whether treatments provided have improved the performance of the heart.

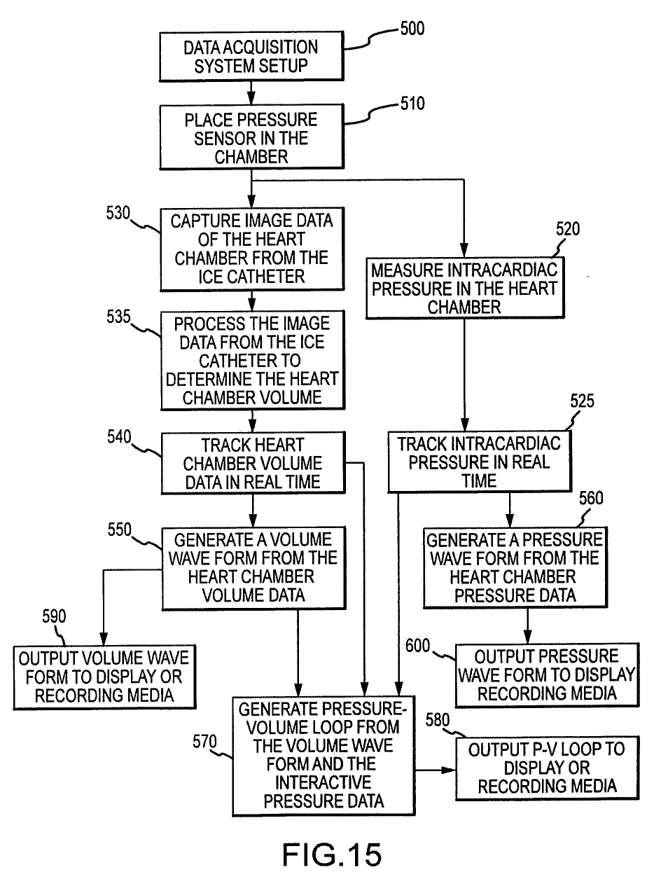

Referring to FIG. 15, a process flow diagram for continuous measurement of cardiac chamber volume with simultaneous collection of intra-cardiac pressure waveform data to implement a PV loop system is described. At step 500 a data system is set up. At step 510 a pressure sensor is placed in the subject chamber. The pressure sensor can be integral with the catheter. At step 520 the pressure senor is activated to measure intracardiac pressure in the heart chamber. Preferably, pressure measurements are continuous in time, but may be made at discrete time intervals. At step 525 the system or apparatus tracks the intracardiac pressure data in real time. At step 530, the system captures image data of the heart chamber, e.g., from the ICE catheter. At step 535, the image data is processed to determine heart chamber volume. At step 540, the heart chamber volume data is tracked in real time.

The data can be output in numerous ways. For example, at step 550, a volume wave form is generated from the heart chamber volume data. At step 590, a wave form of the volume is output to a display or a recording media. Likewise, at step 560, a pressure wave form is generated from the heart chamber pressure data. At step 600, a wave form of the pressure is output to a display or recording media.

Alternatively, at step 570 a PV loop is generated from the volume wave form and the pressure wave form, and at step 580 is output to a display or recording media.

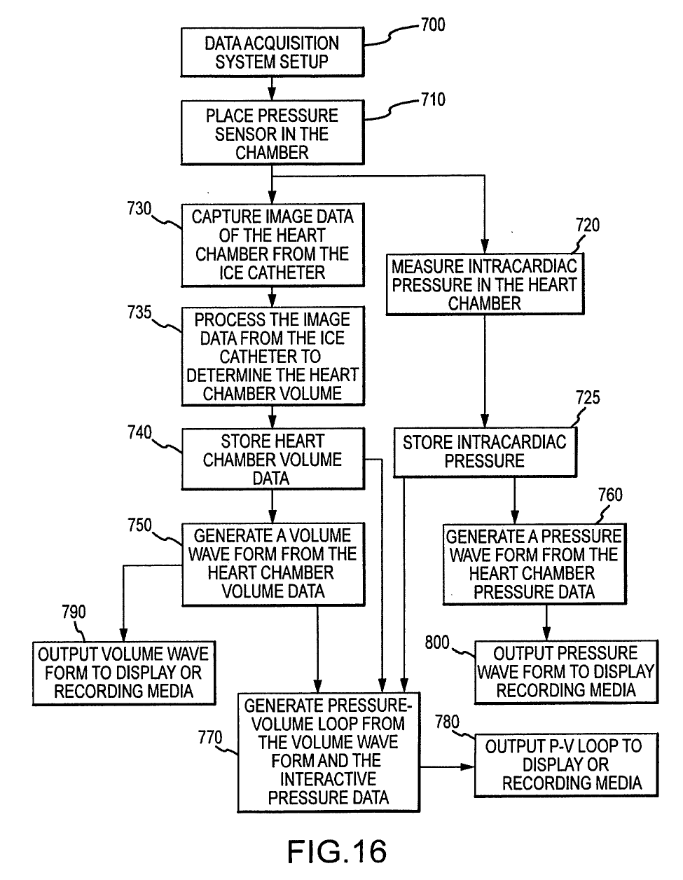

With reference to FIG. 16, in another embodiment, the data is stored and post-processed segmentation and calculation of the data occurs off-line, after the ICE procedure has occurred. At step 700 a data system is set up. At step 710 a pressure sensor is placed in the subject chamber. The pressure sensor can be integral with the catheter. At step 720 the pressure senor is activated to measure intracardiac pressure in the heart chamber. Preferably, pressure measurements are continuous in time, but may be made at discrete time intervals. At step 725 the system or apparatus tracks the intracardiac pressure data in real time and stores it. At step 730, the system captures image data of the heart chamber, e.g., from the ICE catheter. At step 735, the image data is processed to determine heart chamber volume. At step 740, the heart chamber volume data is tracked in real time, which is then stored.

The data can be output in numerous ways. For example, at step 750, after the procedure a volume wave form is generated from the heart chamber volume data. At step 790, a wave form of the volume is output to a display or a recording media. Likewise, at step 760, a pressure wave form is generated from the heart chamber pressure data. At step 800, a wave form of the pressure is output to a display or recording media.

Alternatively, after the procedure, at step 770 a PV loop is generated from the volume wave form and the pressure wave form, and at step 780 is output to a display or recording media.

Although the method and apparatus described above preferably utilize an intracardiac echocardiogram catheter with an integrated pressure sensor, other radiological imaging techniques may be used to derive cardiac anatomy and size in real-time. For instance, transthoracic ultra-sound, TEE (trans-esophageal echocardiography), fluoroscopy, rotational angiography, and MRI mat be used to continuously measure cardiac chamber volume with simultaneous collection of intracardiac pressure wave form data to implement a pressure-volume loop system. The term real-time being as used herein refers to the simultaneous calculation and transformation of data, in whole or part, as it is received from the catheter.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method for generating a pressure-volume loop comprising:
a. capturing image data of the heart chamber;
b. processing the image data to produce segmented heart chamber size data;
c. tracking segmented heart chamber size data;
d. measuring the pressure of the heart chamber;
e. tracking the measured pressure data; and
f. generating a pressure-volume loop from the generated volume data and the measured intracardiac pressure data.

2. The method of claim 1, further comprising placing a pressure sensor in the heart chamber.

3. The method of claim 1, further comprising generating a volume wave form.

4. The method of claim 1, further comprising generating a pressure wave form.

5. The method of claim 2, wherein placing the pressure sensor in the heart chamber comprises guiding a catheter based pressure sensor into the heart chamber.

6. The method of claim 1, wherein the image data is gathered from an intracardiac echocardiographic catheter.

7. The method of claim 6, wherein a pressure sensor is disposed on the intracardiac echocardiographic catheter.

8. The method of claim 7, wherein the pressure sensor comprises a piezoelectric material.

9. The method of claim 7, wherein the pressure sensor comprises a hydrostatic pressure transducer.

10. The method of claim 1, wherein processing captured image data to produce segmented heart chamber size data comprises automatically segmenting the captured image data to provide a direct real time two-dimensional image of cardiac chamber size.

11. The method of claim 1, wherein processing captured image data to produce segmented heart chamber size data comprises automatically segmenting the captured image data to provide a direct real time three-dimensional image of cardiac chamber size.

12. The method of claim 1, wherein processing captured image data to produce segmented heart chamber volume data comprises automatically implementing a snake segmentation method.

13. The method of claim 1, wherein processing captured image data to produce segmented heart chamber volume data comprises automatically implementing a level-set/fast-marching segmentation method.

14. The method of claim 1, further comprising outputting the pressure-volume loop to a display medium in real time.

15. The method of claim 14, further comprising outputting a baseline pressure-volume loop to the display medium.

16. The method of claim 14, further comprising comparing the pressure-volume loop to another pressure-volume loop.

17. The method of claim 16, further comprising evaluating whether the pressure-volume loop represents more efficient hemodynamic performance of the heart than the other pressure-volume loop.

18. A method for generating a pressure-volume loop comprising:
a. capturing geometric data of the heart chamber;
b. processing the geometric data to produce segmented heart chamber size data;
c. tracking segmented heart chamber size data;
d. measuring pressure data in the heart chamber;
e. tracking the measured pressure data; and
f. generating a pressure-volume loop from the generated volume data and the measured intracardiac pressure data.

19. An apparatus for monitoring hemodynamic performance of a cardiac chamber comprising:
a. an intracardiac echocardiogram catheter that comprises
b. a flexible tubular body having a distal end,
c. an ultrasound transducer disposed proximate the distal end, and
d. a pressure sensor disposed in the flexible tubular body such that the pressure sensor is positioned to measure intracardiac pressure when the distal end of the catheter is deployed in the cardiac chamber;
e. a first electrical conductor adapted for electrically connecting the ultrasound transducer to external control circuitry; and
g. a second electrical conductor adapted for electrically connecting the pressure sensor to external control circuitry.

20. The apparatus of claim 19, further comprising control circuitry outside the catheter body such that the control circuitry is electrically coupled to the ultrasound transducer by the first electrical conductor, and the control circuitry is adapted to receive image data signals from the ultrasound transducer.

21. The apparatus of claim 20, wherein the control circuitry is electrically coupled to the pressure sensor by the second electrical conductor, and the control circuitry is adapted to receive intracardiac pressure data signals from the pressure sensor.

22. The apparatus of claim 21, wherein the control circuitry is adapted to generate pressure-volume loop data signals from the surface image data signals and intracardiac pressure data signals in real time.

23. The apparatus of claim 22, further comprising an output medium connected to the control circuitry such that the output medium illustrates hemodynamic performance of the cardiac chamber by displaying a pressure-volume relationship graph from the pressure volume loop data signals generated by the control circuitry.

24. The apparatus of claim 19, wherein the pressure sensor comprises a piezoelectric material.

25. The apparatus of claim 19, wherein the pressure sensor comprises a hydrostatic transducer.

26. The apparatus of claim 22, wherein the control circuitry generates pressure-volume loop data signals from the image data signals and intracardiac pressure data signals in real time by automatically segmenting heart wall surface image data signals to provide a direct real time two-dimensional image of the cardiac chamber size.

27. The apparatus of claim 22, wherein the control circuitry generates pressure-volume loop data signals from the surface image data signals and intracardiac pressure data signals in real time by automatically segmenting heart wall surface image data signals to provide a direct real time three-dimensional image of the cardiac chamber size.

28. The apparatus of claim 22, wherein the control circuitry processes heart wall image data signals to produce segmented heart chamber volume data by automatically implementing a snake segmentation method.

29. The apparatus of claim 22, wherein the control circuitry processes heart wall image data signals to produce segmented heart chamber volume data by automatically implementing a level-set/fast marching method segmentation method.
